SEQUENCE LISTING (2 sequences)

SEQ ID NO: 1 - protein, 247 aa, homo sapiens
MIRRCFQVAARLGHHRGLLEAPRILPRLNPASAFGSSTDSMFSRFLPEKTDLKDYALPNASWCSDMLSLYQEFLEKTKSSGWIKLPSFKSNRDHIRGLKLPSGLAVSSDKGDCRIFTRCIQVEGQGFEYVIFFQPTQKKSVCLFQPGSYLEGPPGFAHGGSLAAMMDETFSKTAFLAGEGLFTLSLNIRFKNLIPVDSLVVMDVEVDKIEDQKLYMSCIAHSRDQQTVYAKSSGVFLQLQLEEESPQ

SEQ ID NO: 2 - protein, 248 aa, Mus musculus
MLRTSFQGVARLVRHKALYRSPCLLPRVHLASAFGSSTESLVARFCPEKTDLKDYALPNASWCSDMLSLYQEFLEKTKSGGWIKLPSFKSNRDHIQGLKLPFGLETASDKQDWRLFTRSIQLEGQGYEYVIFFHPSEKKSVCLFQPGPYLEGAPGFAHGGSLAALMDETYSKTAYLAGEGLFTLSLNIKFKNLIPVGSLAVLDIQVEKIEDQKLYMSCIAQSRDKQTVYAKSSGVFLQLQLEEQSQEQ

(19) United States
(12) Patent Application Publication
(10) Pub. No.: US 2011/0265194 A1
(43) Pub. Date: Oct. 27, 2011

(54) THEM5-MODIFIED MODELS OF NON-ALCOHOLIC FATTY LIVER DISEASE

(76) Inventors: Bettina Duemmler, Kusnacht (CH); Brian Arthur Hemmings, Bettingen (CH); Elena Zhuravleva, Basel (CH)

(21) Appl. No.: 13/126,344

(22) PCT Filed: Oct. 29, 2009
(86) PCT No.: PCT/EP2009/064303

§ 371 Date: Jun. 28, 2011

(30) Foreign Application Priority Data
Oct. 31, 2008 (EP) 08168075.3

(51) Int. Cl. A01K 67/00; C07H 21/00; C12N 1/00; C12N 9/16; C12N 5/10; C12N 15/87; C12N 15/63

(52) U.S. Cl. 800/9; 800/21; 536/23.2; 435/320.1; 435/196; 435/325; 435/419; 435/243

(57) ABSTRACT

The invention provides a new reproducible genetically-modified mouse model for the study of non-alcoholic fatty liver disease. In particular, the invention concerns the study of non-alcoholic fatty liver disease in an THEM5 knockout mouse model and its use in drug discovery and research.

### FIELD OF THE INVENTION

The present invention relates to genetically-modified nonhuman animals useful as an experimental model of non-alcoholic fatty liver disease, wherein the THEM5 gene is altered, producing an animal lacking functional THEM5.

### BACKGROUND OF THE INVENTION

Nonalcoholic (or non-alcoholic) fatty liver disease (NAFLD) refers to a wide spectrum of liver disease ranging from simple fatty liver (steatosis), to nonalcoholic steatohepatitis (NASH), to cirrhosis (irreversible, advanced scarring of the liver). All of the stages of NAFLD have in common the accumulation of fat (fatty infiltration) in the liver cells (hepatocytes). In NASH, the fat accumulation is associated with varying degrees of inflammation and scarring of the liver. NASH is more common in women and the most common cause is obesity. It is also often accompanied by visceral fat distribution, insulin resistance, dyslipidemia and hypertension. NASH can progress to fibrosing, steatohepatitis and trigger cirrhosis, end-stage liver disease and hepatocellular carcinoma. NASH is becoming recognized as the most common cause of liver disease, second only to Hepatitis C in numbers of patients going on to cirrhosis.

In fatty liver, fat accumulates in the liver cells. Simple fatty liver usually does not damage the liver, but is a condition that can be identified by taking a sample of liver tissue (liver biopsy) and examining it under a microscope. Simple fatty liver is not associated with any other liver abnormalities such as scarring or inflammation. It is a common finding in patients who are very overweight or have diabetes mellitus. Alcoholism can also result in inflammation of the liver (alcoholic hepatitis) and/or scarring (alcoholic cirrhosis); it can be differentiated from non-alcoholic liver inflammation by patient history. Possible explanations for fatty liver include the transfer of fat from other parts of the body, or an increase in the extraction of fat presented to the liver from the intestine. Another explanation is that the fat accumulates because the liver is unable to change it into a form that can be eliminated.

Non-alcoholic fatty liver disease (NAFLD) is an important cause of chronic liver disease worldwide. NAFLD is strongly associated with metabolic syndrome and insulin resistance and its prevalence is on the rise. NAFLD represent a spectrum ranging from simple steatosis (SS) to non-alcoholic steatohepatitis (NASH). Accumulating evidence suggests that NASH is potentially progressive, whereas simple steatosis, indicated by liver biopsies, follows a more benign course with little or no progression. NASH is also described as inflammation of the liver associated with the accumulation of fat in the liver, and it differs significantly from the simple accumulation of fat in the liver (fatty liver, or hepatic steatosis) in that the inflammation causes significant damage to the liver cells while simple fatty liver probably does not.

The fatty tissue in the liver may break up liver cells (steatonecrosis) and the patient may develop cirrhosis (scarring of the liver). Recent studies indicate that NASH can result in the development of fibrous tissue in the liver (fibrosis) in up to 40% of patients or cirrhosis in 5-10% of patients. It is not certain why some NASH patients will progress to this serious form of chronic liver disease while others do not. Studies report that the progression to fibrosis or cirrhosis for NASH patients is variable but can occasionally occur in less than 20 years. Some studies have shown that 20% to 40% of people who are grossly overweight will develop NASH. If a patient is grossly overweight, however, it does not mean that he/she will develop NASH.

There are currently no specific therapies for NASH/NAFLD. The most important recommendations given to persons with this disease are to reduce their weight (if obese or overweight), follow a balanced and healthy diet, increase physical activity, avoid alcohol, and avoid unnecessary medications. Experimental approaches under evaluation in patients with NASH include antioxidants, such as vitamin E, selenium, and betaine. Another experimental approach to treating NASH is the use of newer antidiabetic medications—even in persons without diabetes. However, the effectiveness of these drugs is unknown. Thus, a need exists for new treatments for NASH and for experimental models to study and test said new treatments.

A number of animal models with features of the metabolic syndrome including fatty liver have been described. No single model as yet can answer all the research questions. However, each model that resembles NAFLD in onset and course provides insight into mechanisms and consequences (Xirouchakis et al., Current Pharmaceutical Design, 2008, 14(4):378-384). One important issue is that similar to humans, the phenotype of an animal model also depends on its genetic background, gender and age. The metabolic alterations in these models are thus either spontaneous or induced by genetic mutations or by an acquired phenotype after a manipulation, such as high caloric feeding. The genetically mutated models (reviewed in e.g. Anstee & Goldin, Int J Exp Pathol, 2006; 87(1):1-16; London & George, Clin Liver Dis, 2007; 11(1): 55-74; and Koteish & Diehl, Semin Liver Dis, 2001; 21(1): 89-104) are divided in: 1) those with increased lipid import or synthesis, and, 2) those with reduced lipid export or catabolism. The first group includes the following: (a) the ob/ob mouse with leptin deficiency, (b) the db/db mouse, (c) the fa/fa and (d) cp/cp rats with leptin resistance due to leptin receptor deficiency, (e) the CD 36−/− mouse (CD 36 is a fatty acid translocase found in the adipose and muscle tissue) with increased levels of fatty acid and triglycerides in serum which then accumulate within the liver cells, (f) the PEPCK-N SREBP-1a mouse with increased hepatic sterol regulatory element binding protein (SREBP)-1a expression which promotes fatty acid synthase activity and de novo synthesis, and (g) the aP2-N SREBP-1c mouse with SREBP-1a overexpresion in adipose tissue. In the second group, the models are characterized by a defective expression in enzymes regulating β-oxidation such as the following (a) acetyl CoA oxidase−/− mouse, (b) the aromatase (Cyp 19)-deficient mouse, (c) the MTP−/− mouse, (d) the Cyp2e1 knock out mouse. Alternatively there is: (e) a defect in a transcription factor-PPARa−/− mouse which is unable to upregulate β-oxidation or, (f) a defect in transport factors-the juvenile visceral steatosis mouse—in which a mutation in the carnitine transporter gene Octn2 leads to a failure in fatty acid transport into mitochondria for β-oxidation. Models lack of TNFa (TNFa knock out mouse) or its receptor (TNFR1 or TNFR1 and 2 knock out mice), or enzymes induced by TNFa like the C-Jun N-terminal kinase (jnk) 1 or jnk2 knock out mice, as also bile salt export pump (BSEP) the canalicular ATP-dependent bile salt transporter knock out mice, Apo E (a ligand for low density lipoproteins) knock out mice, and inducible nitric oxide synthase (iNOS) knock out mice have also been shown to develop steatosis.

The feeding-induced steatosis models (reviewed in in e.g. Anstee & Goldin, Int J Exp Pathol, 2006; 87(1):1-16; London & George, Clin Liver Dis, 2007; 11(1): 55-74; and Koteish & Diehl, Semin Liver Dis, 2001; 21(1): 89-104) are also divided into 2 groups: those with increased lipid import or synthesis, and those with reduced lipid export or catabolism. In the first group there are the following: (a) the high fat diet, (b) the high sucrose/fructose and (c) high argentine deficient mice. In the second group there are: (a) methionine/choline deficient (MCD) diet (sucrose 40%, fat 10% and no methionine and choline), and, (b) steroids/estrogen/tamoxifen mice. In both models of the second group impaired β-oxidation contributes to the development of steatosis but in the steroids/estrogen/tamoxifen model there is also reduced hepatic triglyceride secretion. Although hepatic lipid accumulation and insulin resistance is present in all animal models of steatosis only few of them progress spontaneously to steatohepatitis and fibrosis (NASH). The diet models which develop steatosis on an MCD diet or a high fat diet develop spontaneously steatohepatitis and fibrosis. On the other hand, in most of the models with genetic alterations steatohepatitis and fibrosis is induced by diet. The ob/ob mouse and the db/db mouse when fed an MCD diet and the fa/fa and cp/cp rats on a high fat (60% lard) diet develop steatohepatitis and fibrosis. The same happens for the knock out mice PPARa, Cyp2el, jnk 1, TNFa and BSEP while on MCD diet, TNFR1 and 2 and OPN on reduced MCD diet, Apo E on a high fat diet, and iNOS on a high fat/high cholesterol/cholic acid diet.

### SUMMARY OF THE INVENTION

The present inventors have now surprisingly found that the inhibition in mice of THEM5, an uncharacterized protein, which was initially cloned by some of the inventors due to its similarity to the CTMP1 protein, leads to symptoms which are similar to the ones observed in human NAFLD.

The present invention hence provides a genetically-modified non-human animal whose somatic and germ cells contain a gene encoding an altered form of an THEM5 gene, the altered THEM5 gene having been targeted to replace a wild-type THEM5 gene into the animal or an ancestor of the animal at an embryonic stage using embryonic stem cells. In some embodiments of the invention, the genetically-modified non-human animal is a mouse.

In some embodiments of the invention, the genetically-modified non-human animal is fertile and is capable of transmitting the altered THEM5 gene to its offspring.

For example, the altered THEM5 gene can be introduced into an ancestor of the genetically-modified non-human animal of the invention at an embryonic stage by electroporation of altered embryonic stem cells. In some embodiments, the altered THEM5 gene can be introduced into the genetically-modified non-human animal at an embryonic stage either by electroporation of altered embryonic stem cells into genetically-modified non-human animal blastocysts or co-incubation of altered embryonic stem cells with fertilized eggs or morulae.

The altered form of THEM5 can be, ins some embodiments of the invention, either nonfunctional or is derived from a species other than said genetically-modified non-human animal, for instance human THEM5.

An ideal use of the genetically-modified non-human animal of the invention is the use as an experimental model for non-alcoholic fatty liver, to identify e.g. new treatments for NASH, and or study its pathogenesis.

The present invention also provides methods of producing a genetically-modified non-human animal whose somatic and germ cells contain a gene encoding an altered form of THEM5. In some embodiments, the altered gene can have been targeted to replace the wild-type THEM5 gene into the genetically-modified non-human animal or an ancestor of said genetically-modified non-human animal at an embryonic stage using embryonic stem cells by a method comprising the steps of (i) introducing a gene encoding an altered form of THEM5 designed to target the THEM5 gene into embryonic stem cells of said genetically-modified non-human animal, for example by electroporation, (ii) injecting the embryonic stem cells containing the altered THEM5 gene into blastocysts of said genetically-modified non-human animal, (iii) transplanting the injected blastocysts into a recipient genetically-modified non-human animal; and (iv) allowing the embryo to develop producing a founder genetically-modified non-human animal mouse.

The present invention also encompasses an isolated nucleic acid molecule comprising, or consisting of, a sequence encoding THEM5, as well as the nucleotide sequence complementary thereto. Moreover, the present invention also encompasses a recombinant vector comprising the nucleic acid of the invention, as well as a host cell comprising said vector.

### BRIEF DESCRIPTION OF THE DRAWINGS

FIG. 1: Loss of THEM5 protein results in activation of PKB after i.v. insulin injection. THEM5 WT and KO animals were starved for 8 hrs and injected into v. cava with 1 U/kg of insulin. Organs for analysis were collected 5 min after insulin injection and processed for WB analysis (loading 50 ug protein/lane).

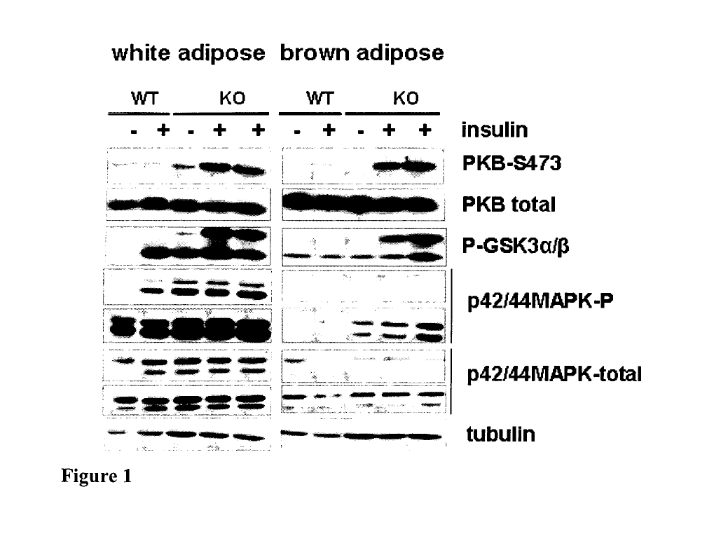

FIG. 2: Increased PKB phosphorylation in primary hepatocytes from THEM5KO mice upon insulin stimulation. Livers of THEM5WT and KO mice were perfused with collagenase solution, and primary hepatocytes were plated into 6-wells plate, starved for 24 hrs and then stimulated with 100 nM insulin for indicated time. Cells were lysed, and analyzed by WB analysis (loading 50 ug protein/lane).

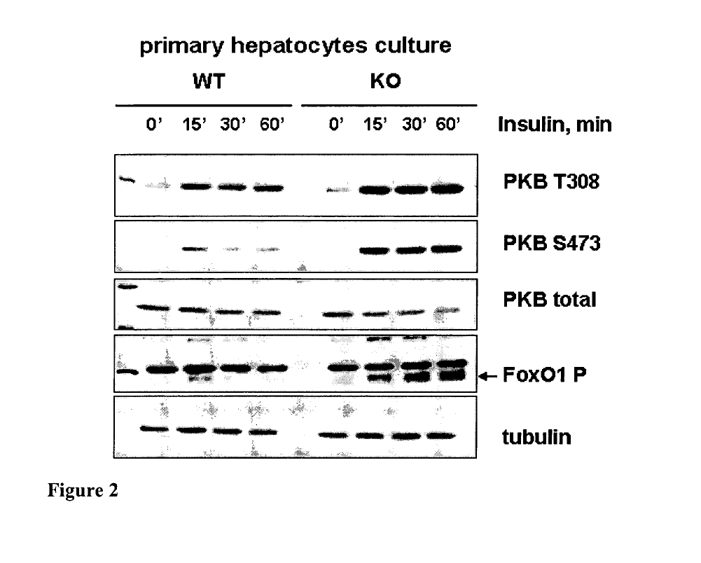

FIG. 3: Increased abdominal fat content in THEM5 HET mice compared to the WT littermate (left).

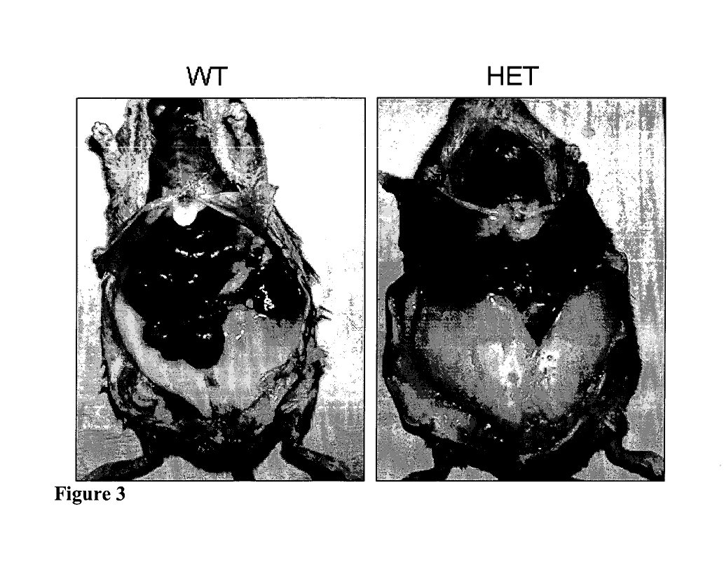

FIG. 4: Oil Red O (upper panel) and hematoxylin/eosin staining (lower panel) of liver from a control (left) and heterozygote (right) female mouse, each 37 weeks of age.

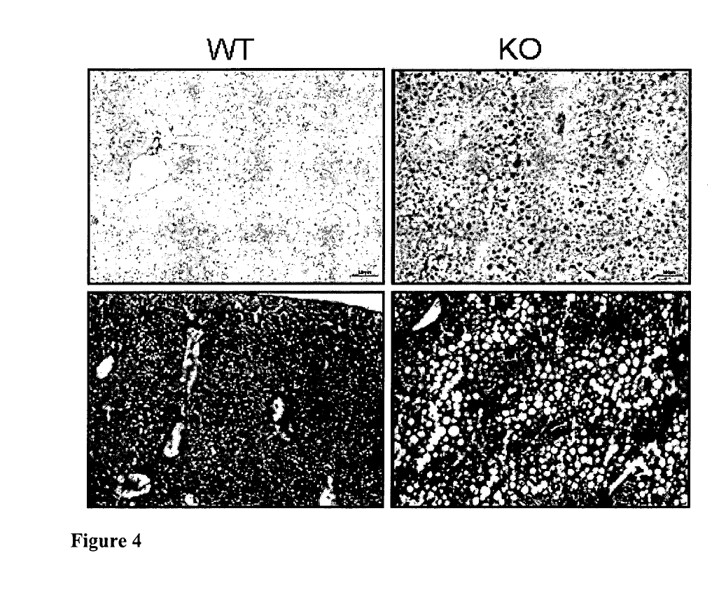

FIG. 5: Phase-contrast picture of primary hepatocytes from WT and KO THEM5 mice.

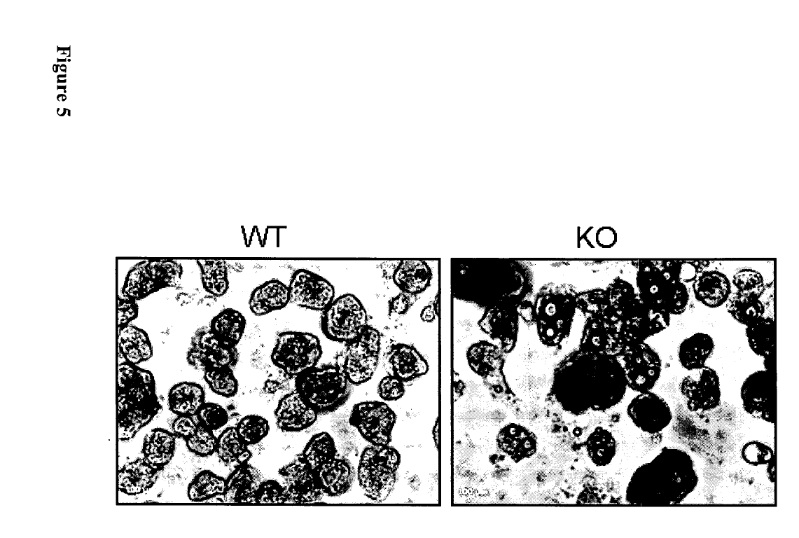

FIG. 6: Oil Red O staining of primary hepatocytes from WT and KO THEM5 mice.

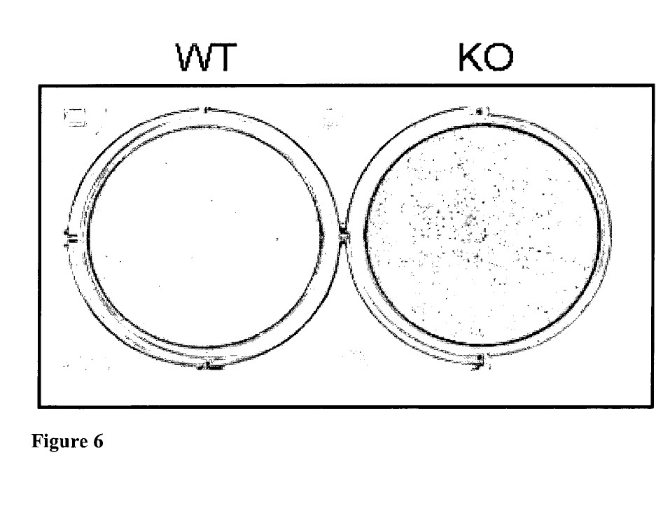

### DETAILED DESCRIPTION OF THE INVENTION

The present inventors have now surprisingly found that the inhibition in mice of THEM5, an uncharacterized protein, which was initially cloned by some of the inventors due to its similarity to the CTMP1 protein, leads to symptoms which are similar to the ones observed in human NAFLD.

The present invention hence provides a genetically-modified non-human animal whose somatic and germ cells contain a gene encoding an altered form of an THEM5 gene, the altered THEM5 gene having been targeted to replace a wild-type THEM5 gene into the animal or an ancestor of the animal at an embryonic stage using embryonic stem cells. In some embodiments of he invention, the genetically-modified non-human animal is a mouse.

In some embodiments of the invention, the genetically-modified non-human animal is fertile and is capable of transmitting the altered THEM5 gene to its offspring.

For example, the altered THEM5 gene can be introduced into an ancestor of the genetically-modified non-human animal of the invention at an embryonic stage by electroporation of altered embryonic stem cells. In some embodiments, the altered THEM5 gene can be introduced into the genetically-modified non-human animal at an embryonic stage either by electroporation of altered embryonic stem cells into genetically-modified non-human animal blastocysts or coincubation of altered embryonic stem cells with fertilized eggs or morulae.

The altered form of THEM5 can be, in some embodiments of the invention, either nonfunctional or is derived from a species other than said genetically-modified non-human animal, for instance human THEM5.

An ideal use of the genetically-modified non-human animal of the invention is the use as an experimental model for non-alcoholic fatty liver, to identify e.g. new treatments for NASH, and or study its pathogenesis.

The present invention also provides methods of producing a genetically-modified non-human animal whose somatic and germ cells contain a gene encoding an altered form of THEM5. In some embodiments, the altered gene can have been targeted to replace the wild-type THEM5 gene into the genetically-modified non-human animal or an ancestor of said genetically-modified non-human animal at an embryonic stage using embryonic stem cells by a method comprising the steps of (i) introducing a gene encoding an altered form of THEM5 designed to target the THEM5 gene into embryonic stem cells of said genetically-modified non-human animal, for example by electroporation, (ii) injecting the embryonic stem cells containing the altered THEM5 gene into blastocysts of said genetically-modified non-human animal, (iii) transplanting the injected blastocysts into a recipient genetically-modified non-human animal; and (iv) allowing the embryo to develop producing a founder genetically-modified non-human animal mouse.

The present invention also encompasses an isolated nucleic acid molecule comprising, or consisting of, a sequence encoding THEM5, as well as the nucleotide sequence complementary thereto. Moreover, the present invention also encompasses a recombinant vector comprising the nucleic acid of the invention, as well as a host cell comprising said vector.

Yet another embodiment of the invention encompasses a kit comprising an isolated nucleic acid molecule of the invention, a recombinant vector of the invention, and/or a host cell of the invention.

Another embodiment of the invention encompasses the use of the THEM5 gene or THEM5 protein as a biomarker for NAFLD, for instance by measuring the concentration of this protein, or the expression levels of he gene, in samples from a subject.

In some embodiments of the present invention, the THEM5 gene clone and the corresponding locus in the genome are used to generate genetically-modified animals in which the THEM5 gene has been made non-functional. The alterations to the naturally occurring gene can be modifications, deletions and substitutions. Modifications and deletions render the naturally occurring gene nonfunctional, producing a “knockout” animal. Substitution of the naturally occurring gene for a gene from a second species results in an animal which produces the gene product of the second species. Substitution of the naturally occurring gene for a gene having a mutation results in an animal which produces the mutated gene product. These genetically-modified animals are critical for therapeutic drug studies, the creation of animal models of human diseases, and for eventual treatment of disorders or diseases associated with human homologue of the THEM5 family as described herein and elsewhere. A genetically-modified animal carrying a disruption or “knockout” of the THEM5 gene is useful for the establishment of a non-human model for diseases involving THEM5, for instance non-alcoholic fatty liver disease.

The sequence of the thioesterase superfamily member 5, THEM5, gene, also known as CTMP2 or C-terminal modulator protein 2, Carboxyl-terminal modulator protein 2 is known (e.g. Entrez Gene ID: 284486 human; 66198 mouse). In one embodiment, the THEM5 genomic DNA can be cloned from a mouse genomic library and is checked to make sure it has the expected characteristics of DNA encoding THEM5 protein. A genetically-modified mouse carrying the disrupted THEM5 gene is generated by homologous recombination of a target DNA construct with the endogenous gene on the chromosome. The genetically-modified mouse carrying the disrupted THEM5 gene does not express functional THEM5 molecules anymore, and is therefore useful in establishing an in vivo model for non-alcoholic fatty liver disease. The THEM5 gene is located close to CTMP1 in humans and mice. Thus, these may be paralogues, possible generated by gene duplication. Both proteins are conserved in higher eukaryotes, but are not present in invertebrates; they share 37% of identical amino acids.

The predicted mitochondrial localization of THEM5 protein was confirmed by in vitro analysis of the human and mouse isoforms. According to the in silico analysis, THEM5 protein possesses a 4HBT domain, which is described to be important for the thioesterase enzymatic activity in several bacterial proteins.

The term “non-human animal” is used herein to include all vertebrate animals, except humans. Examples of non-human animals are mice, rats, cows, pigs, horses, chickens, ducks, geese, cats, dogs, etc. The term “non-human animal” also includes an individual animal in all stages of development, including embryonic and fetal stages. A “genetically-modified animal” is any animal containing one or more cells bearing genetic information altered or received, directly or indirectly, by deliberate genetic manipulation at a sub-cellular level, such as by targeted recombination, microinjection or infection with recombinant virus. The term “genetically-modified animal” is not intended to encompass classical crossbreeding or in vitro fertilization, but rather is meant to encompass animals in which one or more cells are altered by, or receive, a recombinant DNA molecule. This recombinant DNA molecule may be specifically targeted to a defined genetic locus, may be randomly integrated within a chromosome, or it may be extrachromosomally replicating DNA. The term “germ-line genetically-modified animal” refers to a genetically-modified animal in which the genetic alteration or genetic information was introduced into germline cells, thereby conferring the ability to transfer the genetic information to its offspring. If such offspring in fact possess some or all of that alteration or genetic information, they are genetically-modified animals as well.

The alteration or genetic information may be foreign to the species of animal to which the recipient belongs, or foreign only to the particular individual recipient, or may be genetic information already possessed by the recipient. In the last case, the altered or introduced gene may be expressed differently than the native gene, or not expressed at all.

The non-functional THEM5 gene generally should not fully encode the same THEM5 native to the host animal, and its expression product should be altered to a minor or great degree, or absent altogether. However, it is conceivable that a more modestly modified THEM5 will fall within the scope of the present invention.

The genes used for altering a target gene may be obtained by a wide variety of techniques that include, but are not limited to, isolation from genomic sources, preparation of cDNAs from isolated mRNA templates, direct synthesis, or a combination thereof.

A type of target cells for transgene introduction is the ES cells. ES cells may be obtained from pre-implantation embryos cultured in vitro and fused with embryos (Evans et al. (1981), Nature 292:154-156; Bradley et al. (1984), Nature 309:255-258; Gossler et al. (1986), Proc. Natl. Acad. Sci. USA 83:9065-9069; Robertson et al. (1986), Nature 322:445-448; Wood et al. (1993), Proc. Natl. Acad. Sci. USA 90:4582-4584). Transgenes can be efficiently introduced into the ES cells by standard techniques such as DNA transfection using electroporation or by retrovirus-mediated transduction. The resultant transformed ES cells can thereafter be combined with morulas by aggregation or injected into blastocysts from a non-human animal. The introduced ES cells thereafter colonize the embryo and contribute to the germline of the resulting chimeric animal (Jaenisch (1988), Science 240:1468-1474). The use of gene-targeted ES cells in the generation of gene-targeted genetically-modified mice was described 1987 (Thomas et al. (1987), Cell 51:503-512) and is reviewed elsewhere (Frohman et al. (1989), Cell 56:145-147; Capecchi (1989), Trends in Genet. 5:70-76; Baribault et al. (1989), Mol. Biol. Med. 6:481-492; Wagner (1990), EMBO J. 9:3025-3032; Bradley et al. (1992), Bio/Technology 10:534-539).

Techniques are available to inactivate or alter any genetic region to any mutation desired by using targeted homologous recombination to insert specific changes into chromosomal alleles.

As used herein, a “targeted gene” is a DNA sequence introduced into the germline of a non-human animal by way of human intervention, including but not limited to, the methods described herein. The targeted genes of the invention include DNA sequences which are designed to specifically alter cognate endogenous alleles.

One function of the liver is to process fats and proteins from digested food. Fatty liver disease covers a range of conditions where there is a build-up of fat in the liver cells. The liver cells (hepatocytes) normally contain some fat and related fatty chemicals (triglycerides, fatty acids, etc). Excess fat is normally passed out of liver cells, into the bloodstream, and then taken up and stored in fat cells (adipose cells) throughout the body. In fatty liver disease, excess fat builds up in liver cells. This is thought to happen if there is some problem or disruption in the normal processing of fat and related fatty chemicals in the liver cells. Simple fatty liver (also called “hepatic steatosis”) is present when the fat content inside liver cells makes up more than 5-10% of the liver's weight. Simple fatty liver is not associated with serious damage or harm to the liver. Nonalcoholic fatty liver disease (NAFLD), as used herein, refers to a wide spectrum of liver disease ranging from: i) simple fatty liver (steatosis), in which there are fat deposits on the liver; ii) nonalcoholic steatohepatitis (NASH) in which there are fat deposits on the liver along with inflammation and damage of the liver; and iii) cirrhosis in which there is irreversible, advanced scarring of the liver. All of the stages of NAFLD have in common the accumulation of fat (fatty infiltration) in the liver cells (hepatocytes). Fatty liver (steatosis) can progress to nonalcoholic steatohepatitis (NASH). In NASH, the fat accumulation is associated with varying degrees of inflammation and scarring of the liver, and in many cases insulin resistance, dyslipidemia and hypertension. NASH can progress to fibrosing, steatohepatitis and trigger cirrhosis, end-stage liver disease, acute live failure and hepatocellular carcinoma. It most often occurs in people with excess body weight, elevated blood lipids, such as cholesterol and triglycerides, and insulin resistance. The present invention also provides methods of treating acute liver failure using compound identified using the experimental models of the invention. Acute liver failure occurs when the cells in the liver die or become damaged in a short period of time. This causes the liver to fail to work normally and can be fatal. Any progressive liver disease, such as cirrhosis, can result in liver failure. Signs of liver failure include encephalopathy (altered brain function, jaundice, ascites fetor hepaticus and failure of coagulation).

Many people with simple fatty liver have other conditions where fatty liver is a complication. Many cases of simple fatty liver develop in people who drink more alcohol than the recommended limits. Over half of people who drink heavily develop simple fatty liver. In these cases simple fatty liver can progress to alcoholic steatohepatitis. In this condition the excess fat in the liver cells is associated with, or may cause, inflammation of the liver. Alcoholic steatohepatitis, may eventually cause scarring (cirrhosis) of the liver.

Insulin resistance is an impaired metabolic response to our body's own insulin, so that active muscle cells cannot take up glucose as easily as they should. As a result, glucose is prevented from entering the cells and remains in the blood stream. The pancreas tries to keep up with the demand by producing more insulin. Eventually, the pancreas cannot keep up with the body's need for insulin, and excess glucose builds up in the bloodstream. Many people with insulin resistance have high levels of blood glucose and high levels of insulin circulating in their blood at the same time.

People with blood glucose levels that are higher than normal but not yet in the diabetic range have “pre-diabetes” (impaired fasting glucose (IFG) or impaired glucose tolerance (IGT)). Pre-diabetes increases the risk of developing type 2 diabetes.

Prediabetes can be detected by either of the two standard tests currently used to diagnose diabetes. In the fasting plasma glucose test (FPG), a normal fasting blood glucose level is under about 100 mg/dl, and fasting blood glucose in the range of about 100 to about 125 mg/dl indicates impaired fasting glucose (IFG), or prediabetes. A fasting blood glucose level over about 125 mg/dl indicates diabetes. In the oral glucose tolerance test (OGTT), a normal blood glucose would be below about 140 mg/dl; an elevated blood glucose level in the range of about 140 to about 199 mg/dl indicates impaired glucose tolerance, or prediabetes. A blood glucose level of about 200 mg/dl or higher indicates diabetes. Gestational diabetes is a condition in which the glucose level is elevated and other diabetic symptoms appear during pregnancy in a woman who has not previously been diagnosed with diabetes. Diabetic symptoms typically disappear following delivery. Gestational diabetes is caused by blocking effects of other hormones on the insulin that is produced by the pancreas (insulin resistance).

Insulin resistance is also associated with syndrome X. Syndrome X is a combination of metabolic disorders. Specifically, syndrome X, which is also known as the “metabolic syndrome”, “Insulin Resistance Syndrome”, or “metabolic syndrome X”, refers to a groups of risk factors for heart disease that seem to cluster in some people. It is defined as the presence of three or more of the following conditions: i) insulin resistance or glucose intolerance ii) elevated blood pressure iii) elevated triglycerides iv) low levels of HDL (high density cholesterol) cholesterol v) central (abdominal) obesity

Other symptoms of Syndrome X may include prothrombotic state (e.g., high fibrinogen or plasminogen activator inhibitor in the blood) and proinflammatory state (e.g., elevated high-sensitivity C-reactive protein in the blood).

For adults, overweight and obesity ranges are determined by using weight and height to calculate a number called the “body mass index” (BMI). Body Mass Index (BMI) is a number calculated from a person's weight and height, using the formula: weight (kg)/[height (m)]˜(calculation: [weight (kg)/height (m)/height (m)]). With the metric system, the formula for BMI is weight in kilograms divided by height in meters squared. BMI is used because, for most people, it correlates with their amount of body fat. An adult who has a BMI between 25 and 29.9 is considered overweight. An adult who has a BMI of 30 or higher is considered obese.

Hyperlipidemia is an elevation of lipids (fats) in the bloodstream. These lipids include cholesterol, cholesterol esters (compounds), phospholipids and triglycerides. They're transported in the blood as part of large molecules called lipoproteins. When hyperlipidemia is defined in terms of a class or classes of elevated lipoproteins in the blood, the term hyperlipoproteinemia is used. Hypercholesterolemia is the term for high cholesterol levels in the blood. Hypertriglyceridemia refers to high triglyceride levels in the blood. The present invention provides methods for treating all of the above listed diseases, disorders and syndromes using a compound identified using the experimental models of the invention. Effective amounts of such compounds are administered to a subject with one or more of these conditions.

As used herein “treating” includes achieving, partially or substantially, one or more of the following results: partially or totally reducing the extent of the disease, disorder or syndrome (e.g., reducing fat deposits, increasing insulin activity, reducing weight); ameliorating or improving a clinical symptom or indicator associated with the disorder; delaying, inhibiting or preventing the progression of the disease, disorder or syndrome; or partially or totally delaying, inhibiting or preventing the onset or development of disorder. Delaying, inhibiting or preventing the progression of the disease, disorder or syndrome includes for example, delaying, inhibiting or preventing the progression of fatty liver to NASH; delaying, inhibiting or preventing the progression of NASH to cirrhosis, end-stage liver disease and/or hepatocellular carcinoma; and delaying, inhibiting or preventing the progression of pre-diabetes to diabetes.

The present invention also provides a method of screening compounds to identify those which might be useful for treating non-alcoholic fatty liver disease in a subject, for instance by modulating the expression or the protein levels of THEM5, as well as the so-identified compounds.

The present invention hence also provides pharmaceutical compositions for treating non-alcoholic fatty liver disease in a subject, for instance by modulating the expression or the protein levels of THEM5. Such compositions comprise a therapeutically effective amount of an inhibitory compound, and a pharmaceutically acceptable carrier. In a specific embodiment, the term “pharmaceutically acceptable” means approved by a regulatory agency of the Federal or a state government or listed in the U. S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, tale, sodium chloride, driied skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the compound, in some embodiments, in purified form, together with a suitable amount of carrier so as to provide the form for proper administration to the patient. The formulation should suit the mode of administration.

In a preferred embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anaesthetic such as lidocaine to ease pain at the site of the injection.

Generally, the ingredients are supplied either separately or mixed together in unit dosage form, for example, as a dry lyophilized powder or water free concentrate in a hermetically scaled container such as an ampoule or sachet indicating the quantity of active agent.

Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compounds of the invention can be formulated as neutral or salt forms.

Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc. The amount of the compound which will be effective in the treatment, inhibition and prevention of a disease or disorder associated with aberrant expression and/or activity of a polypeptide of the invention can be determined by standard clinical techniques. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each patient's circumstances.

Effective doses may be extrapolated from dose-response curves derived from in vitro or animal model test systems.

In the present invention, “isolated” refers to material removed from its original environment (e.g., the natural environment if it is naturally occurring), and thus is altered “by the hand of man” from its natural state. For example, an isolated polynucleotide could be part of a vector or a composition of matter, or could be contained within a cell, and still be “isolated” because that vector, composition of matter, or particular cell is not the original environment of the polynucleotide. The term “isolated” does not refer to genomic or cDNA libraries, whole cell total or mRNA preparations, genomic DNA preparations (including those separated by electrophoresis and transferred onto blots), sheared whole cell genomic DNA preparations or other compositions where the art demonstrates no distinguishing features of the polynucleotide/sequences of the present invention. Further examples of isolated DNA molecules include recombinant DNA molecules maintained in heterologous host cells or purified (partially or substantially) DNA molecules in solution. Isolated RNA molecules include in vivo or in vitro RNA transcripts of the DNA molecules of the present invention. However, a nucleic acid contained in a clone that is a member of a library (e.g., a genomic or cDNA library) that has not been isolated from other members of the library (e.g., in the form of a homogeneous solution containing the clone and other members of the library) or a chromosome removed from a cell or a cell lysate (e.g. , a “chromosome spread”, as in a karyotype), or a preparation of randomly sheared genomic DNA or a preparation of genomic DNA cut with one or more restriction enzymes is not “isolated” for the purposes of this invention. As discussed further herein, isolated nucleic acid molecules according to the present invention may be produced naturally, recombinantly, or synthetically.

In the present invention, a “secreted” protein refers to a protein capable of being directed to the ER, secretory vesicles, or the extracellular space as a result of a signal sequence, as well as a protein released into the extracellular space without necessarily containing a signal sequence. If the secreted protein is released into the extracellular space, the secreted protein can undergo extracellular processing to produce a “mature” protein. Release into the extracellular space can occur by many mechanisms, including exocytosis and proteolytic cleavage.

“Polynucleotides” can be composed of single-and double-stranded DNA, DNA that is a mixture of single-and double-stranded regions, single-and double-stranded RNA, and RNA that is mixture of single-and double-stranded regions, hybrid molecules comprising DNA and RNA that may be single-stranded or, more typically, double-stranded or a mixture of single-and double-stranded regions. In addition, polynucleotides can be composed of triple-stranded regions comprising RNA or DNA or both RNA and DNA. Polynucleotides may also contain one or more modified bases or DNA or RNA backbones modified for stability or for other reasons. “Modified” bases include, for example, tritylated bases and unusual bases such as inosine. A variety of modifications can be made to DNA and RNA; thus, “polynucleotide” embraces chemically, enzymatically, or metabolically modified forms.

The expression “polynucleotide encoding a polypeptide” encompasses a polynucleotide which includes only coding sequence for the polypeptide as well as a polynucleotide which includes additional coding and/or non-coding sequence.

“Stringent hybridization conditions” refers to an overnight incubation at 42 degree C. in a solution comprising 50% formamide, 5×SSC (750 mM NaCl, 75 mM trisodium citrate), 50 mM sodium phosphate (pH 7.6), 5× Denhardt's solution, 10% dextran sulfate, and 20 μg/ml denatured, sheared salmon sperm DNA, followed by washing the filters in 0.1×SSC at about 50 degree C. Changes in the stringency of hybridization and signal detection are primarily accomplished through the manipulation of formamide concentration (lower percentages of formamide result in lowered stringency); salt conditions, or temperature. For example, moderately high stringency conditions include an overnight incubation at 37 degree C. in a solution comprising 6× SSPE (20× SSPE=3M NaCl; 0.2M NaH2PO4; 0.02M EDTA, pH 7.4), 0.5% SDS, 30% formamide, 100 μg/ml salmon sperm blocking DNA; followed by washes at 50 degree C. with 1× SSPE, 0.1% SDS. In addition, to achieve even lower stringency, washes performed following stringent hybridization can be done at higher salt concentrations (e.g. 5×SSC).

Variations in the above conditions may be accomplished through the inclusion and/or substitution of alternate blocking reagents used to suppress background in hybridization experiments. Typical blocking reagents include Denhardt's reagent, BLOTTO, heparin, denatured salmon sperm DNA, and commercially available proprietary formulations. The inclusion of specific blocking reagents may require modification of the hybridization conditions described above, due to problems with compatibility.

The terms “fragment,” “derivative” and “analog” when referring to polypeptides means polypeptides which either retain substantially the same biological function or activity as such polypeptides. An analog includes a proprotein which can be activated by cleavage of the proprotein portion to produce an active mature polypeptide.

The term “gene” means the segment of DNA involved in producing a polypeptide chain; it includes regions preceding and following the coding region “leader and trailer” as well as intervening sequences (introns) between individual coding segments (exons).

Polypeptides can be composed of amino acids joined to each other by peptide bonds or modified peptide bonds, i.e., peptide isosteres, and may contain amino acids other than the 20 gene-encoded amino acids. The polypeptides may be modified by either natural processes, such as posttranslational processing, or by chemical modification techniques which are well known in the art. Such modifications are well described in basic texts and in more detailed monographs, as well as in a voluminous research literature. Modifications can occur anywhere in the polypeptide, including the peptide backbone, the amino acid side-chains and the amino or carboxyl termini. It will be appreciated that the same type of modification may be present in the same or varying degrees at several sites in a given polypeptide. Also, a given polypeptide may contain many types of modifications. Polypeptides may be branched, for example, as a result of ubiquitination, and they may be cyclic, with or without branching. Cyclic, branched, and branched cyclic polypeptides may result from posttranslation natural processes or may be made by synthetic methods. Modifications include, but are not limited to, acetylation, acylation, biotinylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, denivatization by known protecting/blocking groups, disulfide bond formation, demethylation, formation of covalent cross-links, formation of cysteine, formation of pyroglutamate, formylation, gamma-carboxylation, glycosylation, GPI anchor formation, hydroxylation, iodination, linkage to an antibody molecule or other cellular ligand, methylation, myristoylation, oxidation, pegylation, proteolytic processing (e.g., cleavage), phosphorylation, prenylation, racemization , selenoylation, sulfation, transfer-RNA mediated addition of amino acids to proteins such as arginylation, and ubiquitination. (See, for instance, PROTEINS-STRUCTURE AND MOLECULAR PROPERTIES, 2nd Ed., T. E. Creighton, W. H. Freeman and Company, New York (1993); POSTTRANSLATIONAL COVALENT MODIFICATION OF PROTEINS, B. C. Johnson, Ed., Academic Press, New York, pgs. 1-12 (1983); Seifter et al. , Meth Enzymol 182:626-646 (1990); Rattan et al., Ann NY Acad Sci 663:48-62 (1992).)

A polypeptide fragment “having biological activity” refers to polypeptides exhibiting activity similar, but not necessarily identical to, an activity of the original polypeptide, including mature forms, as measured in a particular biological assay, with or without dose dependency. In the case where dose dependency does exist, it need not be identical to that of the polypeptide, but rather substantially similar to the dose-dependence in a given activity as compared to the original polypeptide (i.e., the candidate polypeptide will exhibit greater activity or not more than about 25-fold less and, in some embodiments, not more than about tenfold less activity, or not more than about three-fold less activity relative to the original polypeptide.)

Species homologs may be isolated and identified by making suitable probes or primers from the sequences provided herein and screening a suitable nucleic acid source for the desired homologue.

“Variant” refers to a polynucleotide or polypeptide differing from the original polynucleotide or polypeptide, but retaining essential properties thereof. Generally, variants are overall closely similar, and, in many regions, identical to the original polynucleotide or polypeptide.

As a practical matter, whether any particular nucleic acid molecule or polypeptide is at least 80%, 85%, 90%, 92%, 95%, 96%, 97%, 98%, 99%, or 100% identical to a nucleotide sequence of the present invention can be determined conventionally using known computer programs. A preferred method for determining the best overall match between a query sequence (a sequence of the present invention) and a subject sequence, also referred to as a global sequence aligmnent, can be determined using the FASTDB computer program based on the algorithm of Brutlag et al. (Comp. App. Blosci. (1990) 6:237-245). In a sequence alignment the query and subject sequences are both DNA sequences. An RNA sequence can be compared by converting U's to T's. The result of said global sequence alignment is in percent identity. Preferred parameters used in a FASTDB alignment of DNA sequences to calculate percent identity are: Matrix=Unitary, k-tuple=4, Mismatch Penalty—1, Joining Penalty—30, Randomization Group Length=0, Cutoff Score=1, Gap Penalty—5, Gap Size Penalty 0.05, Window Size=500 or the length of the subject nucleotide sequence, whichever is shorter. If the subject sequence is shorter than the query sequence because of 5′ or 3′ deletions, not because of internal deletions, a manual correction must be made to the results. This is because the FASTDB program does not account for 5′ and 3′ truncations of the subject sequence when calculating percent identity. For subject sequences truncated at the 5′ or 3′ ends, relative to the query sequence, the percent identity is corrected by calculating the number of bases of the query sequence that are 5′ and 3′ of the subject sequence, which are not matched/aligned, as a percent of the total bases of the query sequence. Whether a nucleotide is matched/aligned is determined by results of the FASTDB sequence alignment. This percentage is then subtracted from the percent identity, calculated by the above FASTDB program using the specified parameters, to arrive at a final percent identity score. This corrected score is what is used for the purposes of the present invention. Only bases outside the 5′ and 3′ bases of the subject sequence, as displayed by the FASTDB alignment, which are not matched/aligned with the query sequence, are calculated for the purposes of manually adjusting the percent identity score. For example, a 90 base subject sequence is aligned to a 100 base query sequence to determine percent identity. The deletions occur at the 5′ end of the subject sequence and therefore, the FASTDB alignment does not show a matched/alignment of the first 10 bases at 5′ end. The 10 impaired bases represent 10% of the sequence (number of bases at the 5′ and 3′ ends not matched/total number of bases in the query sequence) so 10% is subtracted from the percent identity score calculated by the FASTDB program. If the remaining 90 bases were perfectly matched the final percent identity would be 90%. In another example, a 90 base subject sequence is compared with a 100 base query sequence. This time the deletions are internal deletions so that there are no bases on the 5′ or 3′ of the subject sequence which are not matched/aligned with the query. In this case the percent identity calculated by FASTDB is not manually corrected. Once again, only bases 5′ and 3′ of the subject sequence which are not matched/aligned with the query sequence are manually corrected for.

By a polypeptide having an amino acid sequence at least, for example, 95% “identical” to a query amino acid sequence of the present invention, it is intended that the amino acid sequence of the subject polypeptide is identical to the query sequence except that the subject polypeptide sequence may include up to five amino acid alterations per each 100 amino acids of the query amino acid sequence. In other words, to obtain a polypeptide having an amino acid sequence at least 95% identical to a query amino acid sequence, up to 5% of the amino acid residues in the subject sequence may be inserted, deleted, or substituted with another amino acid. These alterations of the reference sequence may occur at the amino or carboxy terminal positions of the reference amino acid sequence or anywhere between those terminal positions, interspersed either individually among residues in the reference sequence or in one or more contiguous groups within the reference sequence.

As a practical matter, whether any particular polypeptide is at least 80%, 85%, 90%, 92%, 95%, 96%, 97%, 98%, 99%, or 100% identical to, for instance, the amino acid sequences shown in a sequence or to the amino acid sequnce encoded by deposited DNA clone can be determined conventionally using known computer programs. A preferred method for determining, the best overall match between a query sequence (a sequence of the present invention) and a subject sequence, also referred to as a global sequence alignment, can be determined using the FASTDB computer program based on the algorithm of Brutlag et al. (Comp. App. Biosci. (1990) 6:237-245). In a sequence alignment the query and subject sequences are either both nucleotide sequences or both amino acid sequences. The result of said global sequence alignment is in percent identity. Preferred parameters used in a FASTDB amino acid alignment are: Matrix=PAM 0, k-tuple=2, Mismatch Penalty—I, Joining Penalty=20, Randomization Group Length=0, Cutoff Score=I, Window Size=sequence length, Gap Penalty—5, Gap Size Penalty—0.05, Window Size=500 or the length of the subject amino acid sequence, whichever is shorter. If the subject sequence is shorter than the query sequence due to N-or C-terminal deletions, not because of internal deletions, a manual correction must be made to the results. This is because the FASTDB program does not account for N-and C-terminal truncations of the subject sequence when calculating global percent identity. For subject sequences truncated at the N-and C-termini, relative to the query sequence, the percent identity is corrected by calculating the number of residues of the query sequence that are N-and C-terminal of the subject sequence, which are not matched/aligned with a corresponding subject residue, as a percent of the total bases of the query sequence. Whether a residue is matched/aligned is determined by results of the FASTDB sequence alignment. This percentage is then subtracted from the percent identity, calculated by the above FASTDB program using the specified parameters, to arrive at a final percent identity score. This final percent identity score is what is used for the purposes of the present invention. Only residues to the N-and C-termini of the subject sequence, which are not matched/aligned with the query sequence, are considered for the purposes of manually adjusting the percent identity score. That is, only query residue positions outside the farthest N-and C-terminal residues of the subject sequence. Only residue positions outside the N-and C-terminal ends of the subject sequence, as displayed in the FASTDB alignment, which are not matched/aligned with the query sequence are manually corrected for. No other manual corrections are to be made for the purposes of the present invention.

Naturally occurring protein variants are called “allelic variants,” and refer to one of several alternate forms of a gene occupying a given locus on a chromosome of an organism. (Genes 11, Lewin, B., ed., John Wiley & Sons, New York (1985).) These allelic variants can vary at either the polynucleotide and/or polypeptide level. Alternatively, non-naturally occurring variants may be produced by mutagenesis techniques or by direct synthesis.

Using known methods of protein engineering and recombinant DNA technology, variants may be generated to improve or alter the characteristics of polypeptides. For instance, one or more amino acids can be deleted from the N-terminus or C-terminus of a secreted protein without substantial loss of biological function. The authors of Ron et al., J. Biol. Chem. 268: 2984-2988 (1993), reported variant KGF proteins having hepanin binding activity even after deleting 3, 8, or 27 amino-terminal amino acid residues. Similarly, Interferon gamma exhibited up to ten times higher activity after deleting 8-10 amino acid residues from the carboxy terminus of this protein (Dobeli et al., J. Biotechnology 7:199-216 (1988)). Moreover, ample evidence demonstrates that variants often retain a biological activity similar to that of the naturally occurring protein. For example, Gayle and co-workers (J. Biol. Chem 268:22105-22111 (1993)) conducted extensive mutational analysis of human cytokine IL-1a. They used random mutagenesis to generate over 3,500 individual IL-1a mutants that averaged 2.5 amino acid changes per variant over the entire length of the molecule. Multiple mutations were examined at every possible amino acid position. The investigators found that “[most of the molecule could be altered with little effect on either [binding or biological activity].” (See, Abstract.) In fact, only 23 unique amino acid sequences, out of more than 3,500 nucleotide sequences examined, produced a protein that significantly differed in activity from wild-type. Furthermore, even if deleting one or more amino acids from the N-terminus or C-terminus of a polypeptide results in modification or loss of one or more biological functions, other biological activities may still be retained. For example, the ability of a deletion variant to induce and/or to bind antibodies which recognize the secreted form will likely be retained when less than the majority of the residues of the secreted form are removed from the N-terminus or C-terminus. Whether a particular polypeptide lacking N-or C-terminal residues of a protein retains such immunogenic activities can readily be determined by routine methods described herein and otherwise known in the art.

“THEM5” is also known as thioesterase superfamily member 5, CTMP2 or C-terminal modulator protein 2, or Carboxyl-terminal modulator protein 2n (e.g. Entrez Gene ID: 284486 human; 66198 mouse). This uncharacterized protein, THEM5, was initially cloned by some of the inventors due to its similarity to the CTMP1 protein. The THEM5 gene is located close to CTMP 1 in humans and mice. Thus, these may be paralogues, possibly generated by gene duplication. Both proteins are conserved in higher eukaryotes, but are not present in invertebrates. CTMP1 and THEM5 share 37% of identical amino acids. The predicted mitochondrial localization of THEM5 protein was confirmed by in vitro analysis of the human and mouse isoforms. According to the in silico analysis, THEM5 protein possesses a 4HBT domain, which is described to be important for the thioesterase enzymatic activity in several bacterial proteins. Our in vitro results show that THEM5 protein acts as a thioesterase toward long chain fatty acid CoA esthers.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the present specification, including definitions, will control. In addition, the materials, methods, and examples are illustrative only and not intended to be limiting.

### EXAMPLES

Mice

Mice deficient in THEM5 were generated by targeted disruption of the THEM5 gene. A targeting vector was generated that contains a 3.7 kb 5′ homology region, an IRES/lacZ/neo cassette, and a 5 kb 3′ homology region. A genomic DNA fragment of about 1.3 kb, including the ATG start codon in exon 1 and the full sequence of exon2, is deleted in the targeting vector. The targeting vector was linearized with NotI and electroporated into 129/Ola ES cells. Screening of ES cell clones was performed by Southern Blotting. DNA was digested with EcoRV and probed with an external probe. An internal probe was then used on NdeI digested DNA for further characterization of ES cell clones positive for homologous recombination. Correctly targeted ES cells were used to generate chimeras. Male chimeras were mated with wild type C57BL/6 females to obtain THEM5 mice, which were intercrossed to produce THEM5 homozygous mutants. Progeny were genotyped for the presence of a targeted allele by multiplex PCR.

Mice were housed according to the Swiss Animal Protection Laws in groups with 12-h dark-light cycles and with free access to food and water. All procedures were conducted with the relevant approval of the appropriate authorities.

In Vivo Insulin Stimulation

For insulin stimulation experiments mice of corresponding genotypes were fasted overnight, terminally anesthetized and injected with 1 U/kg of insulin (human recombinant insulin, Sigma) or vehicle (saline solution) via vena cava inferior for indicated time. Tissues of interest were removed and frozen in liquid nitrogen, followed later by homogenization and lysis (described below).

Primary Hepatocytes Culture

For primary hepatocytes isolation mice were terminally anesthetized and perfused through the portal vein first with 10 ml of basal Hanks solution supplemented with 2.5 mM EGTA, 0.1% glucose and 1% Pen/Strep, and then with 15 ml of basal Hanks solution supplemented with 0.5 mg/ml collagenase type I (Sigma) and 5 mM CaCl₂ (perfusion rate 3.5 ml/min). Then liver was removed, homogenized in non-supplemented Williams E Medium (WEM), cell suspension was filtered through 70 μm nylon filter and washed twice with ice-cold non-supplemented WEM to remove cell debris. Cells were counted and plated 750,000/well (6-well plate) in complete WEM medium (10% fetal calf serum, 100 U penicillin/streptomycin, 2 mM glutamine) into collagen-coated 6-well plates. For insulin stimulation experiments cells were starved for 24 hours (WEM with 100 U penicillin/streptomycin, 2 mM glutamine) and stimulated with 100 nM of insulin for indicated time. For Oil Red O staining cells were kept in complete WEM, then fixed with 4% PFA and stained with Oil Red O.

Western Blot Analysis

Organs or cells were lysed in lysis buffer containing 50 mM Tris-HCl pH7.5, 25 mM sodium florid, 40 mM beta-glycerophosphate, 120 mM sodium chloride, 1% NP-40, and cocktail of protease and phosphatase inhibitors. Protein extracts were separated on 10-8% SDS-PAAG and then transferred to Immobilon-P PVDF membranes (Millipore).

Antibodies against total PKB, phospho-PKB (Ser473, Thr308), phospho-glycogen synthase kinase 3α/β (phospho-GSK3α/β[Ser21/9]), total GSK3β, phospho-FoxOl (Ser256), total MAPK p42/44, phospho-MAPK p42/44 were purchased from Cell Signaling Technologies.

Histology

For histological analysis, organs were dissected and fixed in 4% PFA overnight at 4° C. After dehydration in ethanol, samples were embedded in paraffin. Tissues were cut into 3 μm thick sections and stained with hematoxylin-eosin.

For cryosection organs were fixed in 4% PFA overnight at 4° C., then placed in 30% sucrose solution in PBS for 12 hours, mounted with O.C.T. medium and frozen in isopentane. Tissues were cut into 10 μm thick sections and stained with Oil Red O or hematoxylin/eosin.

1. A genetically-modified non-human animal whose somatic and germ cells contain a gene encoding an altered form of an THEM5 gene, the altered THEM5 gene having been targeted to replace a wild-type THEM5 gene into the animal or an ancestor of the animal at an embryonic stage using embryonic stem cells.

2. The genetically-modified non-human animal of claim 1 wherein said animal is a mouse.

3. The genetically-modified non-human animal of claim 1, wherein said genetically-modified non-human animal is fertile and capable of transmitting the altered THEM5 gene to its offspring.

4. The genetically-modified non-human animal of claim 1, wherein the altered THEM5 gene has been introduced into an ancestor of the genetically-modified non-human animal at an embryonic stage by electroporation of altered embryonic stem cells.

5. The genetically-modified non-human animal of claim 1, wherein the altered THEM5 gene has been introduced into the genetically-modified non-human animal at an embryonic stage either by electroporation of altered embryonic stem cells into genetically-modified non-human animal blastocysts or coincubation of altered embryonic stem cells with fertilized eggs or morulae.

6. The genetically-modified animal of claim 1, wherein said altered form of THEM5 is either nonfunctional or is derived from a species other than said genetically-modified non-human animal.

7. The genetically-modified non-human animal of claim 1, wherein said altered form of THEM5 is human THEM5.

8. (canceled)

9. A method of producing a genetically-modified non-human animal whose somatic and germ cells contain a gene encoding an altered form of THEM5, the altered gene having been targeted to replace the wild-type THEM5 gene into the genetically-modified non-human animal or an ancestor of said genetically-modified non-human animal at an embryonic stage using embryonic stem cells, which comprises:
introducing a gene encoding an altered form of THEM5 designed to target the THEM5 gene into embryonic stem cells of said genetically-modified non-human injecting the embryonic stem cells containing the altered THEM5 gene into blastocysts of said genetically-modified non-human animal;
transplanting the injected blastocysts into a recipient genetically-modified non-human animal; and
allowing the embryo to develop producing a founder genetically-modified non-human animal mouse.

10. (canceled)

11. (canceled)

12. An isolated nucleic acid comprising
(i) a nucleotide sequence encoding THEM5,
(ii) or the nucleotide sequence complementary to the nucleotide sequence of (i).

13. A recombinant vector comprising the nucleic acid of claim 12.

14. A host cell comprising the vector of claim 13.

15. An isolated protein or polypeptide coded by the isolated nucleic acid molecule of claim 12.
